(19) United States
(12) Patent Application Publication
(10) Pub. No.: US 2005/0053678 A1
(43) Pub. Date: Mar. 10, 2005

(54) Methods and compositions for betel nut chewing gum

(76) Inventors: Robert T. Gow, Naples, FL (US); John Pierce, Thousand Oaks, FL (US); George W. Sypert, Naples, FL (US)

Correspondence Address: TROUTMAN SANDERS LLP, BANK OF AMERICA PLAZA, SUITE 5200, 600 PEACHTREE STREET , NE, ATLANTA, GA, 30308-2216, US

(21) Appl. No.: 10/945,109

(22) Filed: Sep. 20, 2004

Related U.S. Application Data
(63) Continuation-in-part of application No. 10/818,439, filed on Apr. 5, 2004.
Application No. 10/818,439 is a continuation-in-part of application No. 10/408,888, filed on Apr. 8, 2003, now abandoned.
Application No. 10/818,439 is a continuation-in-part of application No. 10/408,896, filed on Apr. 8, 2003, now abandoned.
Application No. 10/818,439 is a continuation-in-part of application No. 10/273,981, filed on Oct. 18, 2002.
Application No. 10/273,981 is a continuation-in-part of application No. 10/263,579, filed on Oct. 3, 2002.
(60) Provisional application No. 60/369,889, filed on Apr. 3, 2002.
(60) Provisional application No. 60/326,928, filed on Oct. 3, 2001.

(51) Int. Cl. A61K 35/78; A61K 9/68

(52) U.S. Cl. 424/734; 424/48; 424/769

(57) ABSTRACT

The present invention relates to methods and compositions comprising Areca catechu extracts and Piper betel extracts. Compositions comprise Areca catechu extracts that comprise alkaloid compounds in ratios that are different from the ratios found in native Areca plant material. Methods of the present invention comprise extractions of Areca catechu and Piper betel, making the compositions comprising Areca catechu and Piper betel and methods of providing compositions that mimic the effects in humans of chewing betel quid and have reduced carcinogenicity.

### RELATED APPLICATIONS

This application is a continuation-in-part of U.S. patent application Ser. No. 10/818,439, filed Apr. 5, 2004, which is a continuation-in-part of U.S. patent application Ser. No. 10/408,888, filed Apr. 8, 2003, and a continuation-in-part of U.S. patent application Ser. No. 10/408,896, filed Apr. 8, 2003, and is a continuation-in-part of U.S. patent application Ser. No. 10/273,981, filed Oct. 18, 2002, which is a continuation-in-part of U.S. patent application Ser. No. 10/263,579, filed Oct. 3, 2002, which claims priority to U.S. Provisional Patent Application No. 60/326,928, filed Oct. 3, 2001, and 60/369,889, filed Apr. 3, 2002, each of which is incorporated by reference in its entirety as if specifically set forth herein.

### FIELD OF INVENTION

The present invention relates to compositions comprising Areca catechu and Piper betel in formulations such as chewing gum products, tablets, capsules or lozenges, and methods for making and using the same. More specifically, the present invention is related to methods for making Areca catechu chewing gum, tablet, capsule or lozenge compositions that include extracts of Areca catechu and extracts of Piper betel with characteristics differing from native plant material.

### BACKGROUND OF THE INVENTION

Areca, a type of palm tree also known as Areca catechu, is generally cultivated in India, Southeast Asia, the East Indies, Taiwan and East Africa. The fruit of the Areca tree is a nut containing a single seed having a thin seed coat. The nut of Areca catechu contains several pyridine-derived alkaloids, including arecoline, arecaidine, guvacoline and guvacine that may be as high as 1.7% of the nut's makeup. In nature, the highest single alkaloid component concentration in Areca nut is arecoline that contains a methyl ester functional group.

The Areca nut is known to provide stimulating effects, and it is chewed by almost one-half billion people who seek the stimulating properties of the nut. Betel quid is the combination of Areca catechu nut and Piper betel leaf, and other lesser components, and is the most common use of the Areca plant. Betel quid chewing has been shown to be a major etiologic factor in oral cancer among such users. It has been found that Areca nut tannins and arecoline inhibit the growth of oral mucosal fibroblasts and keratinocytes. See J. F. Morton. Basic Life Sci. 59:739-765, 1992, and N. M. Shivapurkar and S. V. Bhide. Ind. J. Pharmacol. 10(4):257-264, 1978. The effects of long-term use include oral submucosal fibrosis, leukoplakia and oral cancer. Studies have shown that conventional Areca nut extracts induce DNA breaks and unscheduled DNA synthesis and differentiation of oral keratinocytes. Arecoline also displays genotoxic effects.

The autonomic effects of Areca nut on the user include sweating and facial flush. Skin temperature rises and heart rate increases when chewing betel quid. The alkaloids arecoline and arecaidine, although initially causing a brief depressor response, subsequently produce an increase in arterial blood pressure and heart rate. These stimuli are mediated through muscarinic M1 receptors. In animals, arecoline, arecaidine, guvacoline and guvacine are known to possess activity as agonists at muscarinic acetylcholine receptors. Additionally, arecoline has been shown to have indirect effects on catecholamine levels, while arecaidine and guvacine inhibit gamma-aminobutyric acid (GABA) receptor uptake in micromolar concentrations.

The chewing of betel quid has led to increased levels of oral cancers. For example, in the United Kingdom, with its large populations of Southeastern Asians, many of whom routinely chew betel quid, there are over 3,000 new cases of oral cancer and 1,700 deaths annually related the use of betel quid. One combination of betel quid uses the leaf of Piper betel plant as a wrapper for various fillings that include Areca palm nut, as pastes, crushed fragments or shavings, along with tobacco, saffron, slaked lime, and aromatic spices and seeds. Some of these combinations add to the carcinogenicity of betel quid.

What is needed are compositions that provide the desired physiological effects provided by Areca catechu nut that have lowered carcinogens, particularly arecoline and tannins, and concurrently satisfy the need for those who prefer to chew betel quid.

### SUMMARY OF THE INVENTION

The present invention comprises compositions and methods for Areca and Piper betel compositions comprising formulations including chewing gums, tablets, capsules, and lozenges as healthy substitutes for betel quid chewing.

An aspect of the present invention comprises methods of selective extraction of compounds such as tannins from extraction compositions of Piper betel and Areca to yield compositions having a lower risk of cancer than the native plant materials. Another aspect of the present invention comprises compositions that are combinations of compositions of Areca extracts with Piper betel extracts.

An aspect of the present invention comprises methods and compositions comprising Areca catechu and Piper betel. Methods of the invention comprise methods of extraction of compounds from plant source material of Areca catechu and Piper betel, methods of making pharmaceutical or nutriceutical products comprising Areca catechu and Piper betel, and methods of use of the extracted products, and pharmaceutical and nutriceutical products made from extract compositions. Compositions of the present invention comprise extraction products of Areca catechu comprising extracted alkaloid compounds that have altered alkaloid profiles that are not found in natural plant material, combined with compositions comprising compounds isolated from the plant material of Piper betel. An embodiment of the compositions of the present invention comprises compositions comprising an extract of Areca catechu which is higher in the soluble carboxylic acid alkaloids than in the less soluble ester compounds of Areca, in combination with compositions of Piper betel that have at least one phenolic compound, or phenolic compounds in ratios different from those found in native Piper betel plant materials. Compositions of the present invention also comprise pharmaceutical and nutriceutical compositions such as chewing gum formulations containing a combination of extracts of Areca catechu and extracts of Piper betel.

An aspect of the present invention also comprises methods of selective extraction of compounds such as tannins from extracts of Areca or Piper betel to yield compositions that have a lowered risk of cancer than the native plant materials. Another aspect of the present invention comprises compositions comprising extraction products of Areca combined with extraction products of Piper betel having phenolic compound compositions that are not found in native plant material.

Another aspect of the present invention comprises methods for making compositions comprising Areca compositions combined with Piper betel compositions that have a predetermined characteristic, such as phenolic compound ratios that are unlike the phenolic compound ratios of native Piper betel plant materials or a lower amount of tannins compared to the Areca plant materials. The compositions of the present invention comprise Areca compositions combined with Piper betel compositions that have a predetermined characteristic, such as an alkaloid amount, that is unlike that found in the unextracted native plant material and in currently known compositions, such as currently used betel quid products. Compositions having differing predetermined alkaloid amounts allow for the production of Areca compositions combined with Piper betel compositions having differing chemical compound amounts for enhancing or reducing certain physiological effects when the compositions are administered. Embodiments of the compositions provide compositions comprising Areca compositions combined with Piper betel compositions having a lowered amount of tannins compared to the Areca plant materials, thereby reducing the risk of oral, esophageal, gastric and bladder cancers associated with betel quid consumption.

In another aspect, the invention relates to formulations of chewing gum having desired physiological and psychological effects, such as central nervous system stimulation, improved cognition, a sense of well being, anti-nicotinic and anorexic properties, that comprise extracts of Areca catechu and Piper betel that have reduced health risks. The compositions of the present invention can be used to make a combined Areca extract and Piper betel extract in formulations such as a paste, resin, oil or powder, beverage, liquid infusion or decoction, or a dry flowable powder. Such products are processed for many different uses, and some embodiments are made into a fast-dissolve tablet or other orally available delivery vehicle.

The Areca or Piper betel plant material is extracted and the resulting extracted Areca or Piper betel compositions each have predetermined chemical compound amounts and can be in the form of a paste, oil or resin, or other form suitable for use or further processing. Preferably, the extraction methods include using supercritical CO₂ extraction and solvent modifiers such as water and ethyl alcohol. The extracted compositions, having predetermined alkaloid amounts, can then be subjected to further processing steps. Piper betel and Areca compositions produced by such methods have predetermined characteristics, such as chemical compound ratios or profiles, that are unlike those found in the native plant materials and the chemical compound profile can be tailored to meet particular considerations for the final product. The Areca and Piper betel compositions so produced can be used alone, combined to make a unitary composition or used in combination with other compounds or other extracted materials, herbal remedies, pharmaceutical agents, food, dietary supplements, or beverages. The Areca and Piper betel compositions can also be used in treatments of physiological conditions.

### DETAILED DESCRIPTION

The present invention comprises methods and compositions of Areca catechu, specifically the Areca nut, and Piper betel, including the leaf, the inflorescence or other parts of the Piper betel plant that provide the desired chemical compounds. Compositions of the invention comprise compositions resulting from extraction of Areca, compositions resulting from extraction of Piper betel, compositions of extracted Areca compounds that have ratios of compounds that are not found in the native plant material, compositions of Piper betel having compounds in ratios not found in the native material, and also include compositions for chewing gum suitable for administration to humans comprising Areca and Piper betel compounds.

The present invention comprises methods and compositions of Areca catechu, particularly the Areca nut, and Piper betel. As used herein, “Areca” or “Areca catechu” refers to the nut or seed of the Areca catechu palm tree. Methods of the present invention comprise making compositions comprising extracted Areca compositions, which may include both the materials extracted from Areca and the extracted residue, combined with extracted Piper betel compositions, which may include both the materials extracted from Piper betel and the extracted residue. Compositions of the present invention comprise compositions resulting from extraction of Areca, such as compositions of extracted Areca that have ratios of alkaloid compounds that are not found in the native plant material, in combination with Piper betel extract compositions. Suitable methods and compositions of Areca catechu and Piper betel are disclosed in U.S. patent application Ser. Nos. 10/818,439, 10/408,888, 10/408,896, and PCT/US04/010733 and PCT/US02/33385, and the disclosures of each are hereby incorporated by reference in its entirety as if specifically set forth herein.

The present invention comprises extracting the nut of Areca catechu, using extraction steps that include transformation of the arecoline (an alkaloid methyl ester) into arecaidine (an alkaloid carboxylic acid) and guvacoline (an alkaloid methyl ester) into guvacine (an alkaloid carboxylic acid), compositions comprising an Areca extract having decreased percentages of arecoline and guvacoline, and compositions in which lipids and tannins present in native Areca plant material are removed to produce end products that are different from the native plant materials. Such end products exhibit the desired pharmacological activities without the risk of oral cancer associated with Areca ingestion.

The present invention comprises compositions comprising extracts of the nut of the Areca catechu palm combined with compositions comprising extracts of Piper betel. Another aspect of the present invention comprises methods of use of compositions comprising combinations of Areca compositions and Piper betel for the replacement of betel quid as well as for the treatment of fatigue and appetite suppression. More specifically, the present invention comprises methods and compositions of combinations of Areca compositions having alkaloid profiles not found in the native plant material and compositions of Piper betel comprising altered chemical compound profiles. The present invention also comprises methods and compositions of combinations of Areca compositions having alkaloid profiles not found in the native plant and Piper betel compositions, such combination compositions may have substantially reduced levels or amounts of arecoline, guvacoline or tannins. Native plant materials as used herein, include plant materials that may be shredded, ground or powdered after picking and drying, but no extractions, other than incidental water or oil loss, due to the physical manipulation of the plant material, are included. The term “alkaloid profile” or “chemical compound profile” shall mean the ratios of alkaloid compounds or chemical compounds found in either Areca or Piper betel, and the relative amounts of each compound in relation to the other compounds in that plant material. The alkaloid profile or chemical compound profile refers to the amount in grams of each alkaloid compound found in Areca, or specifically chemical compounds, such as, but not limited to, phenolic compounds of Piper betel. The native plant material, that has not undergone extractions to remove any components, would have an alkaloid profile or chemical profile exhibiting the types and amounts of alkaloid compounds or chemical-compounds made by the plant. An altered alkaloid profile, altered chemical compound profile, or an alkaloid profile or chemical compound profile different from that of native plant material, means the ratios of the alkaloid compounds or chemical compounds of the composition are different from the ratios found in the native plant material. For example, in an altered alkaloid profile, the amount of one or more alkaloid compounds may be different or the ratios of one or more alkaloid compounds to the total amount or to other alkaloid compounds are different from that found in native plant material.

Compositions of the present invention comprise compositions of Areca catechu, combined with compositions Piper betel extracts, in formulations such as a paste, powder, or in other forms, for use in dietary supplements. Combination compositions may comprise unextracted Areca compositions, or compositions that mimic native Areca plant material alkaloid profiles, with extracted Piper betel compositions, or may be extracted Areca compositions combined with unextracted Piper betel, or compounds that mimic native Piper betel. Various combinations of components and compositions taught herein are contemplated by the present invention. The compositions can be processed to produce consumable items, for example, by mixing it in a food product or in a capsule, or providing the paste itself for use as a dietary supplement, with sweeteners and flavors added as appropriate. Accordingly, such supplements may include, but are not limited to, compositions of Areca combined with Piper betel for oral delivery in the form of chewing gums, tablets, capsules, lozenges, liquids, and emulsions. A dry, flowable powder formulation is also contemplated by the present invention. Other aspects of compositions of the present invention comprise Areca catechu compositions combined with Piper betel extract compositions in the form of a rapid-dissolve tablet.

The present invention comprises compositions comprising extracts of Areca. Methods of making such extracts are taught in patents applications cited herein and by other methods known to those skilled in the art. Compositions of Areca may comprise the alkaloids of the Areca nut. Methods of extracting Areca may include steps comprising supercritical fluids extraction. Other extraction steps may include the transformation of arecoline (an alkaloid methyl ester) into arecaidine (an alkaloid carboxylic acid), and the transformation of guvacoline (an alkaloid methyl ester) to guvacine (an alkaloid carboxylic acid).

The extracted Areca compound compositions have characteristics that are different from the native plant material. For example, an aspect of the compositions of the present invention includes compositions comprising an Areca catechu extract having an altered alkaloid ratio. For example, compositions comprising an Areca catechu extract having a decreased percentage of arecoline and a decreased percentage of guvacoline, when compared to native plant material, are contemplated. Other compositions comprise alkaloid ratios between the methyl ester alkaloids and the carboxylic acids that are different from native plant materials.

The Areca nut, like many nuts, contains a high level of lipids. These lipids are a complicating factor in the extraction of water-soluble compounds from Areca nuts. The Areca nut comprises approximately 0.2% to 1.7% by weight alkaloid compounds. Of that amount, approximately 40-85% is arecoline, 10 to 40% is arecaidine and guvacine is 2 to 30%. Other alkaloids present include guvacoline and areaolidine. For example, a measurement of the nut reveals that the total alkaloid content is approximately 1.14% mass of the dried nut, and of that alkaloid content, approximately 26.5% is guvacine, 25.6% is arecaidine, and 47.9% is arecoline. Other compounds present in the nut include tannins, which are water soluble compounds that comprise about 20%, by weight, of the nut.

One method of extracting the desired alkaloid compounds from the nut comprises a solvent extraction step comprising extracting a dried powder of Areca nut with water at about 10° C. to 80° C., for approximately 15 minutes to approximately 150 minutes, and preferably at least 60 minutes. A pH adjustment step may occur during or after the solvent extracting step to convert the ester alkaloid compounds into carboxylic acid alkaloid compounds. This extraction step yields an extraction product composition comprising alkaloids, tannins and a small amount of lipids. Tannins are removed from this composition by the addition of adsorbents, such as fining with albumin, activated charcoal, or by anion exchange resins. After removal of the tannins, a pH adjustment step may occur. A pH adjustment step, used for converting ester compounds into carboxylic acid compounds comprises adjusting the pH to at least a pH of 12, optionally in the presence of a reducing agent, such as ascorbic acid, and at a temperature of 50° C., for a time period of not less than 15 minutes. The pH converts the ester compounds into the carboxylic acid compounds, such as arecoline to arecaidine. The amount of conversion can be determined by measuring the initial content of the ester compounds, such as arecoline, and then adding the amount of base necessary to convert the desired percentage of the ester compounds, arecoline, to the carboxylic compound, arecaidine. After the conversion, the pH is then lowered to approximately pH 6-8. The pH step may also be used to convert guvacoline to guvacine.

An elevation of the pH above a value of 12.0 in the presence of the nascent fats derived from the Areca catechu nut will result in the production of free fatty acids derived from such fats. To preclude this from happening, pH adjustments upwards are preferably performed after the removal of the fats. This extraction step can yield an extraction product composition comprising alkaloids, tannins, and a low amount of lipids, but in the event that Areca seed derived fats are still present, they can be removed by any one of a variety of means such as chilling and skimming the fat off the surface of the liquid. Tannins are removed from this composition by the addition of absorbents, such as protein precipitation (albumen fining), activated charcoal, anion exchange resin, or precipitation with calcium hydroxide.

Another process for extracting arecoline, arecaidine, guvacoline and guvacine from the Areca catechu comprises supercritical extraction. The Areca catechu nut is isolated from the plant, dried, and then ground into a powder. The powder is then dissolved in alcohol and subjected to supercritical CO₂ extraction procedure. The pressure and temperature are stabilized from between about 200 bar to about 600 bar and about 20° C. to about 70° C. The resulting extracted Areca material may be in a paste, oil, or resin form and is collected. The spent supercritical extractant can either be recycled for future use or vented into the atmosphere. The extractant-to-feed ratio (kg of extractant versus kg of Areca catechu) may range from about 5:1 to about 100:1.

Alternatively, the process of defatting the Areca catechu nut, before a subsequent water extraction process is initiated, can be accomplished by processing the nut feedstock with carbon dioxide in the liquid state, a liquid non-polar organic solvent such as n-hexane under atmospheric pressure, or by using a compressible gas such as propane, or refrigerant gases in the liquid state. This defatting step can be performed before an extraction of the alkaloids and a pH change.

The Areca extract composition of the present invention may be produced by several methods. In one method, the Areca nut is ground and then undergoes alcohol solvent extraction, and the solvent, containing extracted areca compounds, is freeze-dried. The solid material is discarded, or can be used for other extractions. The freeze-dried material then undergoes SFE (Supercritical Fluids Extraction) to substantially remove the lipid compounds, leaving the alkaloids and other water-soluble compounds in the residue. The residue is then dissolved in water to solubilize the tannins, alkaloids and other water-soluble compounds, and the tannins are removed by protein precipitation, resins or other known methods. The aqueous solution, which contains substantially no tannin compounds, is then freeze-dried or concentrated by known methods, and is referred to herein as an Areca extract composition.

The Areca extract material is then suspended in water and mixed vigorously for a period between about 10 minutes to about 60 minutes to produce and maintain micron-sized particles. The temperature of the water may be from room temperature to about 70° C to facilitate efficient mixing and ester cleavage. A suitable chemical base is added to raise the pH of the aqueous solution to a pH of between about 8.0 to about 12.0. The pH of the solution is then held for a period of time between about 15 minutes to about 2.5 hours. The pH of the aqueous solution is then returned to a neutral pH using a suitable acid.

Compositions of the present invention comprise compositions resulting from the extraction of Areca catechu nut. The Areca and Piper betel compositions include both the extract product resulting from extractions methods and the residue from the extraction, including plant material that was extracted and intermediary extracted residues from subsequent extractions. Areca extract compositions comprise extracted products that have an altered alkaloid profile that is different from the native plant material. An aspect of Areca extract compositions of the present invention comprises compositions that have an alkaloid profile of more carboxylic acid alkaloids than ester alkaloids. For example, compositions of the present invention comprise extracts of Areca nut that have a higher percentage of arecaidine than arecoline, and compositions that comprise a higher percentage of guvacine than guvacoline. Compositions of the present invention also comprise alkaloids, tannins and a small amount of lipids. Such compositions may or may not comprise alkaloids wherein the carboxy alkaloids are found in a higher percentage than ester alkaloid compounds. Compositions of the present invention also comprise the residue of extracted Areca nut from which at least one compound has been removed, such as tannins, alkaloids or some lipids. Accordingly, an aspect of the present invention comprises compositions comprising the residue of the extracted Areca nut that is substantially free of tannins, guvacoline, or arecoline, or comprise residues wherein tannins, guvacoline and arecoline are substantially reduced from the amounts found in unextracted Areca plant material, or comprise residues wherein tannins, guvacoline and arecoline are less than 10% of the amounts found in unextracted Areca plant material.

Compositions of the present invention comprise Areca extract compositions comprising alkaloid compounds. Such alkaloids include, but are not limited to, arecoline, arecaidine, areaolidine, guvacine, and guvacoline. Compositions also comprise other compounds, including but not limited to caffeine, theobromine, and theophylline, and herbs or extractions of herbs or other plant materials such as extracts of kava, chocolate, sage, sage oil, guarana, muira puama, and maca.

The values, such as compound identity and amount, of the alkaloid make-up for a sample of an Areca catechu extract can be determined using conventional analytical techniques, such as high performance liquid chromatography and/or gas chromatography or any other analytical technique known to one of ordinary skill in the art.

The present invention further comprises Areca extract compositions wherein the amount of carboxy acid alkaloid compounds is greater than the amount of ester alkaloid compounds, compositions wherein the amount of ester alkaloid compounds is less than that of native Areca plant material, compositions wherein the amount of arecoline is less than the amount of arecaidine, compositions wherein the amount of guvacoline is less than the amount of guvacine, and compositions wherein the amount of arecoline and guvacoline is less than the amount of arecaidine and guvacine. Compositions of the present invention comprise Areca extract compositions wherein the arecoline content is from approximately 0% to approximately 99% of the arecaidine content. Compositions also comprise Areca extract compositions wherein the ester alkaloid content is from approximately 0% to approximately 99% of the carboxy acid alkaloid content. Compositions of the present invention also comprise Areca extract compositions wherein the guvacoline content is from approximately 0% to approximately 99% of guvacine content.

The compositions of the present invention comprise extracted Areca compositions taught herein combined with extracted Piper betel compositions. Compositions also comprise compounds that are found in Piper betel and compounds that are found in Areca that are admixed to form compositions that provide the desired effects when ingested. Extracted Piper betel compositions comprise compositions comprising oils extracted from Piper betel, such as the leaves or inflorescences of Piper betel, compositions comprising phenolic compounds extracted from Piper betel, compositions comprising one or more phenolic compounds extracted from Piper betel, compositions comprising at least one phenolic compound extracted from Piper betel, compositions comprising chavibetol, compositions comprising chavicol, compositions comprising cadinene, and compositions comprising chavibetol, chavicol and cadinene wherein the ratio of chavibetol to chavicol is different than the ratio found in native plant material. Piper betel compositions may comprise compounds that are soluble in aqueous solutions or organic solutions. Piper betel compositions comprise compounds found in Piper betel, whether extracted from Piper betel plant material or provided by chemical synthesis of such compounds. Areca catechu compositions comprise compounds found in Areca catechu, whether extracted from Areca catechu or provided by chemical synthesis of such compounds.

The present invention comprises methods and compositions for the extraction of compounds from the Piper betel plant, particularly the volatile oils obtained from Piper betel, which are combined with one or more of the Areca compositions taught herein. The Piper betel oils yield the warm aromatic taste of the betel leaves and have antiseptic, stimulant and carminative properties. Extracts of Piper betel leaves include, but are not limited to, Piper betel oils, phenolic compounds including, but not limited to, chavibetol, (2-methoxy-3-allylphenol) chavicol (4-allylphenol) and cadinene. The volatile oil can be obtained from the Piper betel leaves or inflorescence via the process of steam distillation of the plant material or by liquid extraction techniques such as using dichloromethane or petroleum ether as the extracting solvent. A further technique that can be used by those skilled in the art is to extract the oil from the plant material using carbon dioxide in the liquid or supercritical state, or by using a liquefied gas such as propane or a refrigerant such as tetrafluoroethane (S-134a).

For example, to obtain the oil from the leaf or the inflorescence of Piper betel and phenolic chemical compounds such as chavibetol and chavicol, one skilled in the art can use a distillation process such as, but not limited to, steam or vacuum distillation and obtain the oil as a distillate. Alternatively, an extraction method can be utilized such as supercritical CO₂ extraction, or an extraction method that utilizes non-polar solvents such as n-hexane, or an extraction method that utilizes a compressible gas such as propane or a refrigerant such as R-134a in the liquid state. In these processes to obtain the oil of the leaf or inflorescence of Piper betel, the leaf or inflorescence can be dried to remove any excess water and the material can be chopped to a tea cut size so as to increase the bulk density of the material, which provides for better packing in the extraction vessel. Methods for distillations are known for applications of the heat and/or steam to the Piper betel plant material so as to volatize the desired phenolic constituents and then these components are collected as a condensate (distillate). Alternatively, an extraction of the Piper betel botanical leaf or inflorescence material can be performed in an enclosed vessel that can be pressurized to up to 5000 psi in the case of CO2 extraction process or up to 100s of psi in the case of a compressible gas such as propane or the refrigerant R-134a. In these latter extraction processes, in addition to the extraction vessel, a collection vessel is required to collect the eluate from the botanical and from which the liquefied gas is recovered as a vapor so as to be used again after being compressed into a liquid. Once all of the liquefied gas is recovered, then the phenolic oil that is derived from Piper betel can be recovered in its entirety. Such phenolic oil is essentially free of all other plant constituents though some small amount of chlorophyll may be present.

An embodiment of the invention is the formulation of compositions of the Areca nut and the Piper betel leaf for oral administration. The present invention comprises compositions and methods for making and using such compositions, where the compositions comprise oral delivery dosage formulations of the extracts of Piper betel and areca nut compositions taught herein. An aspect of the present invention comprises a rapid dissolve tablet, comprising Piper betel compounds and an Areca catechu extract having an alkaloid profile wherein the carboxy acid alkaloid compounds are in a higher concentration than the ester alkaloid compounds. Other embodiments comprise oral dosage forms such as chewing gum, lozenges, pastilles, and tablets.

Such nutraceutical compositions comprise the extracted compositions taught herein. Such compositions can be used in treatments of humans or animals that provide stimulation to the central nervous system, an increased sense of well-being, improved cognition, reduce cravings for people habituated to nicotine, and reduce the appetite. The compositions comprising compounds extracted from Areca nut and Piper betel leaf can be delivered in the oral dosage forms of chewing gum, tablets, capsules, or lozenges. These composition include, but not limited to the following formulations, compositions comprising (1) the alkaloids arecaidine and guvacine; (2) the alkaloid arecoline is in a amount that is less than that of arecaidine and (3) the phenols chavicol (4-allylphenol) and chavibetol (2-methoxy-3-allylphenol) that can be derived from the oil of Piper betel. The compositions can comprise extracted alkaloids such that the sum of the amount of the alkaloids arecaidine and guvacine is an amount of between 0.5 mg and 30 mg per dose. The compositions can also comprise an amount of alkaloid arecoline that is between 0.1 mg and 10.0 mg per dose. The compositions can also comprise an amount of the phenol chavicol in a range from about 0.1 mg to about 100 mg. Compositions can also comprise an amount of the phenol chavibetol in amounts between 0.1 mg and 100 mg.

An embodiment of the present invention comprises a chewing gum formulation, known to those skilled in the art, that comprises the alkaloids arecaidine and guvacine in a range of between 0.5 mg and 30 mg, and chavicol or chavibetol, or a combination of chaivcol and chavibetol in a range of between 0.1 mg and 100 mg. Such chewing gum formulations also comprise compositions of Areca extracts comprising arecaidine and guvacine in ranges from 1.0 mg to 20 mg, from 2.0 mg to 18 mg, from 5 to 10 mg, from 0.5 mg to 5 mg of arecaidine and 0.5 to 5 mg guvacine. Such chewing gum formulations comprise Piper betel oil compounds in ranges of 1.0 mg to 300 mg, chavibetol from 0.1 mg to 20 mg and chavicol 0.5 mg to 50 mg. Particular formulations comprise chewing gum formulations wherein arecaidine compounds are from 0.5 mg to 15 mg, guvacine from 0.5 mg to 15 mg, chavibetol 0.5 mg to 20 mg and chavicol 1.0 mg to 50 mg.

Thus, in one embodiment, a composition comprises the extracted compositions taught herein in a chewing gum formulation. The formulations of chewing gum are conventional, and well-known to those skilled in the art. For example, a carrier may be provided that may be mixed with the extract compositions. Suitable carriers, particularly in formulating chewing gums, comprise Arabic, guar, and natural rubber gums. Other typical components are sweeteners (sugar, saccharin, sorbitol, aspartame), flavoring agents (e.g., mints, fruits, spices, coloring agents, and the like. For example, the chewing gum or solid carrier may be composed, in its basic formula, of ingredients such as sucrose, corn syrup, gum base, coloring, and flavoring. Ingredients such as HSH (hydrogenated starch hydrolysate), sorbitol, xylitol, and/or isomalt can replace sucrose and corn syrup at different ratios.

As an example of preparation, the chewing gum or solid carrier may be composed, in its basic formula, of ingredients such as sucrose, corn syrup, gum base, softeners such as soy lecithin, coloring and flavoring, ingredients such as HSH (hydrogenated starch hydrolysate), sorbitol, xylitol, and/or isomalt can replace sucrose or corn syrup at different ratios. For example, to a hot water jacketed stainless steel gum mixer equipped with sigma tangential blades rotating at 9-12 rpm with a 1:2 rotating ratio, molten gum base may be added at approximately 55-60° C., and corn syrup or HSH, added at room temperature in the desired amounts, and mixed until fully dispersed. When a homogenous mix is obtained, sucrose or sorbitol, xylitol, isomalt, or a softener such as soy lethicin may be added, all in powdered form, and mixed until fully dispersed. During the process of the addition of the powdered material, the extract compositions are added. Color, flavoring, and any other ingredient deemed necessary for the particular formula may also be added. The gummy mass is then discharged from the gum mixer and conveyed to the gum forming equipment. The addition of flavoring is optional and can be added to prepare distinctly different chewing gums. Conventional chewing gum processing technology involves melting a gum base in, for example, a sigma blender, and adding components such as the extracts of Areca and Piper betel, and sweeteners and flavorants to the melt. The melted mass is then extruded, rolled into sheets, and cut to the desired shape on the rollers. Other processes for making gum oral dosage forms are known. For example, U.S. Pat. No. 4,000,321 to Mochizuki et al. is directed to a process for preparing chewing gum, in which a chewing gum composition is cooled to −15° C. to facilitate fragmentation, and the cooled composition is pulverized with a crusher, hammer mill, pelletizer or turbomill. The pulverized product is then melted to cause the pulverized pieces to co-adhere, forming a chewing gum having low specific gravity and a soft chewing texture. U.S. Pat. No. 5,711,961 to Reiner et al. discloses a pharmaceutical chewing gum composition in tablet form made by freezing chewing gum, grinding the gum in a mill, and granulating the ground gum in a fluid bed. In the current invention, the Areca and Piper betel compounds are then mixed with the granulate, and the granulates are compressed into tablets. In U.S. Pat. No. 4,975,270 to Kehoe, a medicament-active chewing gum is disclosed which is made by freezing and grinding into a particle mass an elastomer, an active agent such as the Areca and Piper betel compositions taught herein, and silica in the presence of liquefied carbon dioxide. The particles are then shaped into a chewing gum product. In the process of Kehoe, the gum and the extracted products from Areca and Piper betel are mixed together while heating, and then the mixture is frozen and ground into particles. In U.S. Pat. No. 4,753,805 to Cherukuri et al. a chewing gum composition in the form of a tablet having a low moisture content is disclosed. The tablet is produced by grinding a chewing gum composition, granulating the ground composition, blending the granulated composition with the compositions taught herein and a compression aid, and compressing the granulated product to form a tablet.

The foregoing description includes the best presently contemplated mode of carrying out the invention. This description is made for the purposes of illustrating the general principles of the inventions and should not be taken in a limiting sense. This invention is further illustrated by the following examples, which are not to be construed in any way as imposing limitations upon the scope thereof. On the contrary, it is to be clearly understood that resort may be had to various other embodiments, modifications, and equivalents thereof, which, after reading the description herein, may suggest themselves to those skilled in the art without departing from the spirit of the present invention.

### EXAMPLES

### Example 1

Steps in the extraction of Areca catechu:
1. Grind dried Areca catechu nut to a particle size of 50 mesh or ¼″ minus.
2. Defatting the ground Areca material using supercritical CO₂ (6000 psi, 50 degree C.) or another appropriate method (see above).
3. Hot water (>75 degree C.) to extract the alkaloids
4. Remove tannins using albumen precipitation (100 mg albumen/liter)
5. Change pH to 12 using NaOH to transform arecholine into arecaidine
6. Change pH to 7 (neutral) using HCl
7. Freeze dry extract
   Alkaloids by % dry weight=6%
      Arecaidine=47%
      Arecoline=15%
      Guvacine=36%
         Steps in the extraction of Piper betel:
1. Dry Piper betel leaf and chop into “tea cut”
2. Steam distillation of Piper betel material to obtain volatile oils
3. Collect volatile oil (1.5% of original dry mass)
   Piper betel oil
      Chavicol=11%
      Chavibetol=5%
A chewing gum formulation is made, and comprises a chewing gum base having by weight: sucrose 10-80%, corn syrup 5-60%, gum base 10-90%; sorbitol 10-60%; hydrogenated starch hydrolysate 5-60%; hydrolyzed proteins 1-8%; isomalt 10-80%, xylitol 10-80%; and artificial sweeteners 0.2-2.0%, are mixed together by melting the gum base in a sigma blender and adding the above ingredients along with an amount of areca extract and Piper betel extract such that the resulting pieces of gum, or individual dosage forms comprise areca extract of approximately 30 mg of arecaidine and guvacine, and approximately 50 mg of chavicol or chavibetol or a combination of the two.

Persons chew, also referred to herein as ingest, 1 to 20 pieces of gum per day. The chewing releases the extracted compounds and the person feels stimulated, or an increased sense of well being, thinks better, or has a reduced appetite. Other persons, who are addicted to nicotine and are not currently ingesting nicotine, have relief from nicotine cravings.

### Example 2

The following ingredients were mixed for the following chewing gum formulation.

| | | |
|---|---|---|
| Extract of Areca catechu | | 100 mg |
| Arecaidine | 4.1 mg | |
| Arecoline | 3.1 mg | |
| guvacine | 3.6 mg | |
| Extract of Piper betel (oil) | | 100 mg |
| chavicol | 10.2 mg | |
| Chavibetol | 4.9 mg | |
| Gum base | | 1000 mg |
| Sucrose | | 500 mg |
| Corn syrup | | 200 mg |
| Soy lethicin | | 60 mg |
| Red lake 40 (coloring) | | 10 mg |
| BHT | | 20 mg |
| Mocha flavor | | 10 mg |
| Total | | 2000 mg |

Although the above formulation was made into a chewing gum form, other chewing gum formulations can be made with varying doses of the active ingredients (extracts of Areca catechu and Piper betel). Similarly, the active ingredient can be formulated in other oral delivery forms such as tablets, capsules, lozenges, etc. and administered as needed daily or up to 20 times per day as needed for the person to feel stimulated, enhanced feeling of well-being, improved cognition and reduction in appetite. Other individuals, who are addicted to nicotine and are currently not ingesting nicotine, have relief from nicotine craving.

1. A composition, comprising, a combination of an Areca catechu extract with an altered alkaloid profile and a Piper betel extract.

2. The composition of claim 1, wherein the Areca catechu extract comprises a greater amount of carboxy acid alkaloid compounds than the amount of ester alkaloid compounds.

3. The composition of claim 1, wherein the amount of ester alkaloid compounds is less than that of native Areca catechu.

4. The composition of claim 1, wherein the amount of arecoline is less than the amount of arecaidine, and the amount of guvacoline is less than the amount of guvacine.

5. The composition of claim 1, wherein the Piper betel extract comprises chavibetol, chavicol or a combination of chavicol and chavibetol.

6. The composition of claim 1, wherein the Areca extract comprises arecaidine and guvacine in an amount of from 0.5 mg to 30 mg, and the Piper betel extract comprises chavibetol in an amount from 5 mg to 50 mg, and chavicol in an amount from 5 mg to 50.

7. An oral dosage form composition comprising a delivery vehicle and a composition comprising a combination of an Areca catechu extract with an altered alkaloid profile and a Piper betel extract.

8. The composition of claim 7, wherein the delivery vehicle comprises a chewing gum base, a tablet, capsule, lozenge, liquid or emulsion.

9. The composition of claim 7, wherein the Areca catechu extract comprises a greater amount of carboxy acid alkaloid compounds than the amount of ester alkaloid compounds.

10. The composition of claim 7, wherein the amount of ester alkaloid compounds is less than that of native areca catechu.

11. The composition of claim 7, wherein the amount of arecoline is less than the amount of arecaidine, and the amount of guvacoline is less than the amount of guvacine.

12. The composition of claim 7, wherein the Piper betel extract is chavibetol, chavicol or a combination of chavicol and chavibetol.

13. The composition of claim 8, wherein the Areca extract comprises arecaidine and guvacine in an amount of from 0.5 mg to 30 mg, and the Piper betel extract comprises chavibetol in an amount from 5 mg to 50 mg, and chavicol in an amount from 5 mg to 50.

14. A method of providing the physiological aspects of chewing betel quid, comprising administering to a human an oral dosage form composition comprising a delivery vehicle and a combination of an Areca catechu extract with an altered alkaloid profile and a Piper betel extract.

15. The method of claim 14, wherein the Areca catechu extract comprises a greater amount of carboxy acid alkaloid compounds than the amount of ester alkaloid compounds.

16. The method of claim 14, wherein the amount of ester alkaloid compounds is less than that of native Areca catechu.

17. The method of claim 14, wherein the amount of arecoline is less than the amount of arecaidine, and the amount of guvacoline is less than the amount of guvacine.

18. The method of claim 14, wherein the Piper betel extract is chavibetol, chavicol or a combination of chavicol and chavibetol.

19. The method of claim 14, wherein the areca extract comprises arecaidine and guvacine in an amount of from 0.5 mg to 30 mg, and the Piper betel extract comprises chavibetol in an amount from 5 mg to 50 mg, and chavicol in an amount from 5 mg to 50.
